(19) United States
(12) Patent Application Publication
(10) Pub. No.: US 2012/0109567 A1
(43) Pub. Date: May 3, 2012

(54) SYSTEMS AND METHODS FOR ACQUIRING AND MANAGING SENSOR DATA RELATED TO DISSOLUTION TESTING APPARATUS

(76) Inventors: Anna Bobasheva, Raleigh, NC (US); Deon Smit, Cary, NC (US); George Bryan Crist, Wilmington, NC (US)

(21) Appl. No.: 12/938,735

(22) Filed: Nov. 3, 2010

(51) Int. Cl. G06F 19/00; G01N 33/15

(52) U.S. Cl. 702/108

(57) ABSTRACT

In acquiring and managing measurement data relating to operating parameters of a dissolution tester, operating parameters are measured by operating one or more sensors. The measured operating parameters are transmitted from the sensors to a user computing device. It is then determined whether the measured operating parameters are in compliance or non-compliance with one or more standards, by comparing the measured operating parameters with a plurality of corresponding predefined values. The measured operating parameters and indications of compliance or non-compliance of each measured operating parameter are stored as a data record in a memory local or remote to the user computing device. The data record may be accessible by a computing device remote from the memory. The operating parameters may include, for example, shaft parameters.

### FIELD OF THE INVENTION

The present invention relates generally to dissolution testing of analyte-containing media. More particularly, the present invention relates to the acquisition and management of sensor data relating to physical or mechanical parameters associated with the operation of dissolution testing apparatus.

### BACKGROUND OF THE INVENTION

Dissolution testing is often performed as part of preparing and evaluating soluble materials, particularly pharmaceutical dosage forms (e.g., tablets, capsules, and the like) containing therapeutically active drug compounds carried by excipient materials. Typically, dosage forms are dropped into test vessels that contain dissolution media of a predetermined volume and chemical composition. For instance, the composition may have a pH factor that emulates a gastro-intestinal environment. Dissolution testing can be useful, for example, in studying the drug release characteristics of the dosage form or in evaluating the quality control of the process used in forming the dosage unit. As a dosage form is dissolving in the test vessel of a dissolution system, optics-based measurements of samples of the solution may be taken at predetermined time intervals through the operation of analytical equipment such as a spectrophotometer. The analytical equipment may determine analyte (e.g. active drug) concentration and/or other properties. The dissolution profile for the dosage form under evaluation—i.e., the percentage of analytes dissolved in the test media at a certain point in time or over a certain period of time—can be calculated from the measurement of analyte concentration in the sample taken. Dissolution media samples may be pumped from the test vessel(s) to a sample cell contained within the spectrophotometer, scanned while residing in the sample cell, and in some procedures then returned to the test vessel(s). Alternatively, a fiber-optic “dip probe” may be inserted directly in a test vessel. The dip probe includes a sample cell and one or more optical fibers that communicate with the spectrophotometer.

To ensure validation of the data generated from dissolution-related procedures, dissolution testing is often carried out according to guidelines or regulations specified by certain entities such as United States Pharmacopeia (USP), the United States Food and Drug Administration (FDA), etc., in which case the dissolution testing apparatus must operate within various parametric ranges. The parameters may include, for example, dissolution media temperature, the amount of allowable loss of evaporated or volatile media from test vessels, vessel plate levelness, dissolution testing apparatus vibration, and the use, position and speed of agitation devices, dosage-retention devices, and other instruments operating in the test vessels, for example, shaft centering and verticality, shaft rotational speed, shaft wobble, and basket wobble.

For instance, the apparatus utilized for carrying out dissolution testing typically includes a vessel plate having an array of apertures into which test vessels are mounted. When the procedure calls for heating the media contained in the vessels, a water bath is often provided underneath the vessel plate such that each vessel is at least partially immersed in the water bath to enable heat transfer from the heated bath to the vessel media. Alternatively, heating elements may be attached directly to the vessel. For any given vessel, the temperature of the media must be maintained at a prescribed temperature (e.g., 37+/−0.5° C.) if certain USP dissolution methods are being conducted. In one exemplary type of test configuration (e.g., USP-NF Apparatus 1), a cylindrical basket is attached to a metallic drive shaft and a pharmaceutical sample is loaded into the basket. One shaft and basket combination is manually or automatically lowered into each test vessel mounted on the vessel plate, and the shaft and basket are caused to rotate. In another type of test configuration (e.g., USP-NF Apparatus 2), a blade-type paddle is attached to each shaft, and the pharmaceutical sample is dropped into each vessel such that it falls to the bottom of the vessel. When proceeding in accordance with the general requirements of Section <711> (Dissolution) of USP24-NF19, each shaft must be positioned in its respective vessel so that that shaft's centerline is not more than 2 mm at any point from the vertical axis of the vessel, and such that the paddle or basket mounted at the lower end of the shaft is positioned at 25 mm+/−2 mm from the bottom of the vessel.

A dissolution testing apparatus should itself be tested periodically to ensure that it is operating within the physical parameters required to meet the relevant standards of the FDA, USP, or the like. Conventionally, physical measurements are made on a dissolution testing apparatus with the use of multiple manual devices and/or electronic sensors. Such devices and sensors are stand-alone devices, examples of which are the VK 5010™ product and QAII™ product commercially available from Varian, Inc., Palo Alto, Calif. See, e.g., VK5010 Centerline Height Measurement System Operator's Manual and QAII C Station Operator's Manual, both available online at www.varianinc.com, the contents of which are hereby incorporated in their entireties. Hence, the physical measurements are made with no centralized control or centralized capture of information, and with no means for reporting or managing the totality of information available from a single dissolution tester or multiple dissolution testers. Moreover, the information is typically captured by hand and transcribed into notebooks, or directly printed upon capture and the printout affixed to a page in the notebook. Many of the steps of the procedure for evaluating a dissolution testing apparatus and subsequent reporting are done manually, leading to human error.

Accordingly, there is a need for a system that provides a greater degree of automation of the acquisition of measurement data associated with operating dissolution testers. There is also a need for a system that acquires and manages measurement data for one or more dissolution testers in a centralized manner.

### SUMMARY OF THE INVENTION

To address the foregoing problems, in whole or in part, and/or other problems that may have been observed by persons skilled in the art, the present disclosure provides methods, processes, systems, apparatus, instruments, and/or devices, as described by way of example in implementations set forth below.

According to one implementation, a method is provided for acquiring and managing measurement data relating to operating parameters of a dissolution tester. The dissolution tester may be of the type that includes a vessel support plate, a plurality of vessels mounted to the vessel support plate, a drive unit, and a plurality of shafts extending from the drive unit, movable by the drive unit into the respective vessels, and rotatable by the drive unit. Operating parameters are measured by operating respective sensors. The measured operating parameters are transmitted from the sensors to the user computing device. It is then determined whether the measured operating parameters transmitted to the user computer device are in compliance or non-compliance with one or more standards, by comparing the measured operating parameters with a plurality of corresponding predefined values. The measured operating parameters and indications of compliance or non-compliance of each measured operating parameter are stored as a data record in a memory.

According to another implementation, a system for acquiring and managing measurement data relating to operating parameters of a dissolution tester includes the dissolution tester, a plurality of sensors, a memory, and a user computing device in signal communication with the dissolution tester and with the sensors. The sensors are configured for operative coupling to the dissolution tester for measuring a plurality of respective operating parameters of the dissolution tester. The user computing device is configured for performing an evaluation that includes: (a) receiving a plurality of measured operating parameters generated by the respective sensors; (b) determining whether the measured operating parameters are in compliance or non-compliance with one or more standards, by comparing the measured operating parameters with a plurality of corresponding predefined values; and (c) storing the measured operating parameters and indications of compliance or non-compliance of each measured operating parameter as a data record in the memory.

Other devices, apparatus, systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims.

### BRIEF DESCRIPTION OF THE DRAWINGS

The invention can be better understood by referring to the following figures. The components in the figures are not necessarily to scale, emphasis instead being placed upon illustrating the principles of the invention. In the figures, like reference numerals designate corresponding parts throughout the different views.

FIG. 1 is a perspective view of an example of a dissolution test apparatus of the type for which physical parameters are measured and the resulting data managed according to an implementation of the present disclosure.

FIG. 2 is a diagrammatic view of an example a system for acquiring and managing data relating to the physical parameters of a dissolution tester such as that shown in FIG. 1, according to the present teachings.

### DETAILED DESCRIPTION OF THE INVENTION

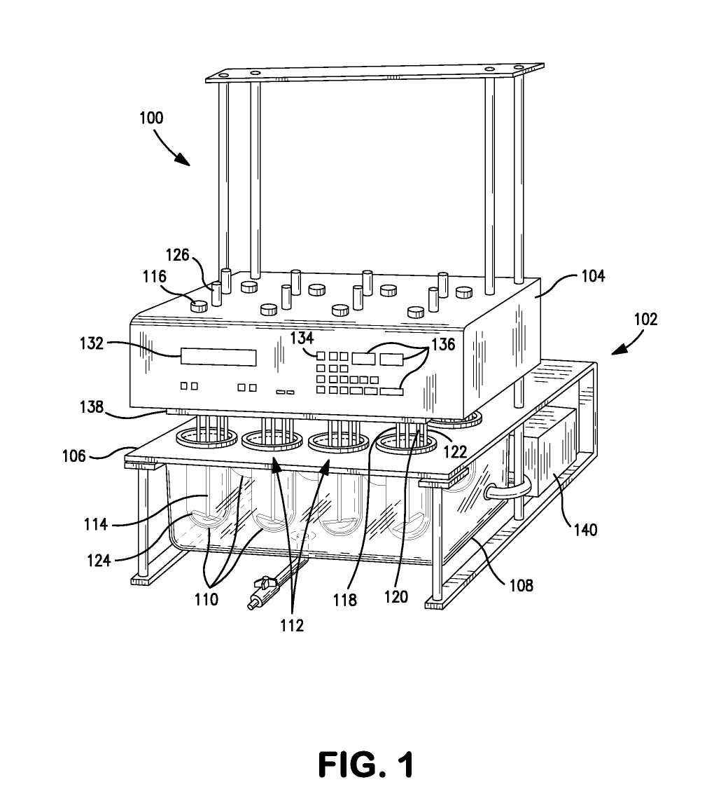

FIG. 1 is a perspective view of an example of a dissolution test apparatus (or dissolution tester) 100 of the type for which physical parameters are measured and the resulting data managed according to an implementation of the present disclosure. The dissolution tester 100 may include a frame assembly 102 supporting various components such as a main housing, drive unit or head assembly 104, a vessel support member (e.g., a plate, rack, etc.) 106 below the drive unit 104, and a water bath container 108 below the vessel support member 106. The vessel support member 106 supports a plurality of vessels 110 extending into the interior of the water bath container 108. FIG. 1 illustrates eight vessels 110 by example, but it will be understood that more or less vessels 110 may be provided. The vessels 110 may be centered in place on the vessel support member 106 at a plurality of vessel mounting sites 112 with the use of vessel centering rings or other suitable means. Vessel covers (not shown) may be provided to prevent loss of media from the vessels 110 due to evaporation, volatility, etc. Optionally, the vessel covers may be coupled to the drive unit 104 and movable by motorized means into position over the upper openings of the vessels 110, as disclosed for example in U.S. Pat. No. 6,962,674, assigned to the assignee of the present disclosure. Water or other suitable heat-carrying liquid medium may be heated and circulated through the water bath container 108 by means such as an external heater and pump module 140, which may be included as part of the dissolution tester 100. Alternatively, the dissolution tester 100 may be a waterless heating design in which each vessel 110 is directly heated by some form of heating element disposed in thermal contact with the wall of the vessel 110, as disclosed for example in U.S. Pat. Nos. 6,303,909 and 6,727,480, assigned to the assignee of the present disclosure.

The drive unit 104 may include mechanisms for operating or controlling various components that operate in the vessels 110 (in situ operative components). For example, the drive unit 104 typically includes a motor, linkages, chucks and other mechanisms for supporting and rotating shafts 114 that operate in each vessel 110. Depending on the procedure being undertaken, paddles 124 or dosage unit-containing baskets (not shown) may be attached to the respective shafts 114. Individual clutches 116 may be provided to alternately engage and disengage power to each shaft 114 by manual, programmed or automated means. The drive unit 104 may also include mechanisms for operating or controlling media transport cannulas that provide liquid flow paths between liquid lines and corresponding vessels 110. Accordingly, the media transport cannulas may include media dispensing cannulas 118 for dispensing media into the vessels 110 and media aspirating cannulas 120 for removing media from the vessels 110. The media dispensing cannulas 118 and the media aspirating cannulas 120 communicate with a pump assembly (not shown) via fluid lines (e.g., conduits, tubing, etc.). The pump assembly may be provided in the drive unit 104 or as a separate module supported elsewhere by the frame 102 of the dissolution tester 100, or as a separate module located external to the frame 102. The pump assembly may include separate pumps for each media dispensing line and/or for each media aspirating line. The pumps may be of any suitable design, one example being the peristaltic type.

The drive unit 104 may also include mechanisms for operating or controlling other types of in situ operative components 122 such as fiber-optic probes for measuring analyte concentration, temperature sensors, pH detectors, dosage form holders (e.g., USP-type apparatus such as baskets, nets, cylinders, etc.), video cameras, etc. A dosage delivery module 126 may be utilized to preload and drop dosage units (e.g., tablets, capsules, or the like) into selected vessels 110 at prescribed times and media temperatures. Additional examples of mechanisms for operating or controlling various in situ operative components are disclosed for example in above-referenced U.S. Pat. No. 6,962,674. The drive unit 104 may also include a programmable systems control module for controlling the operations of various components of the dissolution tester 100 such as those described above. Peripheral elements may be located on the drive unit 104 such as an LCD display 132 for providing menus, status and other information; a keypad 134 for providing user-inputted operation and control of spindle speed, temperature, test start time, test duration and the like; and readouts 136 for displaying information such as RPM, temperature, elapsed run time, vessel weight and/or volume, or the like.

The dissolution tester 100 may further include one or more movable components for lowering operative components 114, 118, 120, 122 into the vessels 110 and raising operative components 114, 118, 120, 122 out from the vessels 110. The drive unit 104 may itself serve as this movable component. That is, the entire drive unit 104 may be actuated into vertical movement toward and away from the vessel support member 106 by manual, automated or semi-automated means. Alternatively or additionally, other movable components such as a driven platform 138 may be provided to support one or more of the operative components 114, 118, 120, 122 and lower and raise the components 114, 118, 120, 122 relative to the vessels 110 at desired times.

In a typical operation, each vessel 110 is filled with a predetermined volume of dissolution media by pumping media to the media dispensing cannulas 118 from a suitable media reservoir or other source (not shown). One of the vessels 110 may be utilized as a blank vessel and another as a standard vessel in accordance with known dissolution testing procedures. Dosage units are dropped either manually or automatically into one or more selected media-containing vessels 110, and each shaft 114 and corresponding paddle 124 (or other agitation or USP-type device) is rotated within its vessel 110 at a predetermined rate and duration within the test solution as the dosage units dissolve. In other types of tests, shaft 114 is attached to a cylindrical basket or cylinder (not shown) instead of a paddle 124, as noted previously. Each basket or cylinder is loaded with a dosage unit and is rotated or reciprocated within the test solution. Media temperature is maintained by immersion of each vessel 110 in the water bath of water bath container 108, or alternatively by direct heating as described previously. The rotating speed of the shafts 114 may also be maintained for similar purposes. The various operative components 114, 118, 120, 122 provided may operate continuously in the vessels 110 during test runs. Alternatively, the operative components 114, 118, 120, 122 may be lowered manually or by an automated assembly 104 or 138 into the corresponding vessels 110, left to remain in the vessels 110 only while sample measurements are being taken at allotted times, and at all other times kept outside of the media contained in the vessels 110. During a dissolution test, sample aliquots of media may be pumped from the vessels 110 via the media aspiration cannulas 120 and conducted to an analyzing device (not shown) such as, for example, a spectrophotometer to measure analyte concentration from which dissolution rate data may be generated. In some procedures, the samples taken from the vessels 110 are then returned to the vessels 110 via the media dispensing cannulas 118 or separate media return conduits. Alternatively, sample concentration may be measured directly in the vessels 110 by providing fiber-optic probes as appreciated by persons skilled in the art. After a dissolution test is completed, the media contained in the vessels 110 may be removed via the media aspiration cannulas 120 or separate media removal conduits.

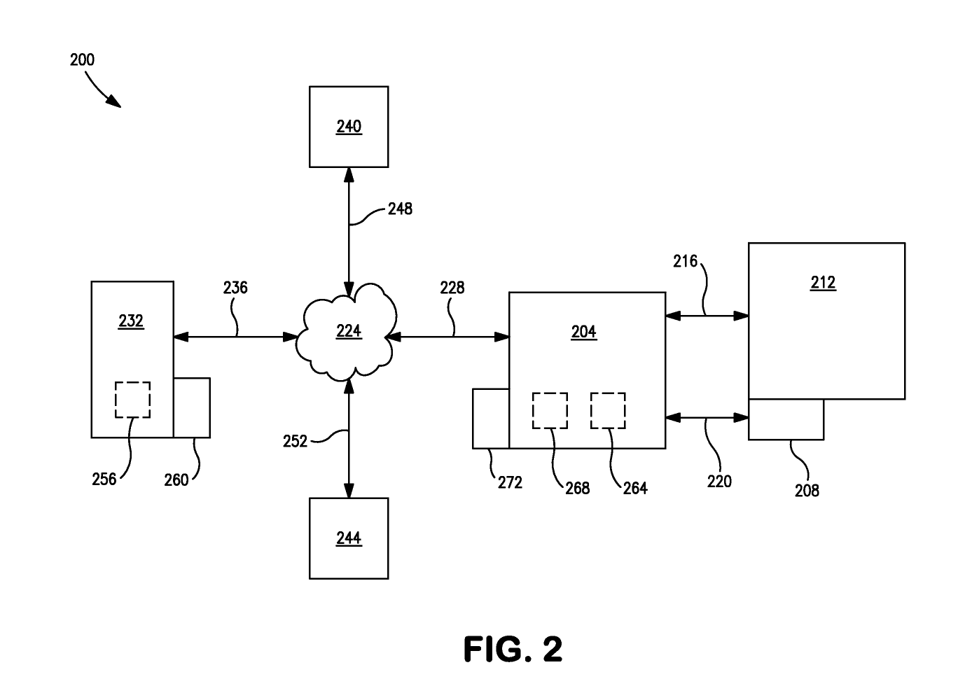

FIG. 2 is a diagrammatic view of an example of a system 200 for acquiring and managing data relating to the physical parameters of a dissolution tester such as that shown in FIG. 1, according to the present teachings. The system 200 includes a local user computing device 204 and one or more sensors 208 communicating with a dissolution tester 212. The user computing device 204 may be any suitable computing device such as, for example, a stationary computer (e.g., a desktop or floor-mounted computer), a portable computer (e.g., a laptop or notebook computer), or a handheld computer (e.g., a tablet computer, a personal digital assistant, a cellular telephone, etc.). Accordingly, the user computing device 204 may include the types of hardware, firmware and software components typically associated with personal computers as appreciated by persons skilled in the art. The user computing device 204 may, for example, include electronic processors (e.g., central processing unit, arithmetic logic unit, digital signal processor, application specific integrated circuit, input and output interfaces, digital communication interfaces, etc.), memory units (e.g., system memory, non-volatile memory, volatile memory, hard drive, removable storage media, etc.), data busses, power regulation circuitry, input devices (e.g., keyboard or keypad, mouse or other pointing device, touch screen, microphone, voice-recognition device, etc.), output devices (e.g., display, monitor, printer, strip-chart recorder, sound-producing device, etc.), and so on. The user computing device 204 may include an operating system such as, for example, Microsoft Windows® software, for controlling and managing the various functions of the user computing device 204. The user computing device 204 may also include software for displaying a graphical user interface to facilitate interface between the user and the user computing device 204.

The user computing device 204 is “local” in the sense that it is typically operated in the same room as the dissolution tester 212 and its communication with the dissolution tester 212 does not require a network interface shared by other computing devices. The user computing device 204 may communicate with the dissolution tester 212 over any communication link 216 suitable for carrying data. The communication link 216 may be wired (e.g., cable, transmission line, optical fiber, etc.) or wireless (e.g., radio frequency, cellular telephony, infrared, etc.) as appreciated by persons skilled in the art. For example, the user computer device 204 and the dissolution tester 212 may include respective RS232 ports for establishing a wired communication link 216 (e.g., a cable). As another example, the user computer device 204 and the dissolution tester 212 may include respective radio frequency (RF) transmission and receive circuitry for establishing a wireless communication link 216. In some implementations, the user computer device 204 communicates with the drive unit 104 (FIG. 1) of the dissolution tester 212 over the communication link 216 so as to control the shafts 114 of the dissolution tester 212 during acquisition of certain physical measurement data of the dissolution tester 212.

The sensors 208 in FIG. 2 are a diagrammatic representation of one or more different types of sensors that may be coupled to the dissolution tester 212 by a user to measure different types of physical parameters. Some types of sensors may be provided as a group in an integrated sensor unit, and thus the sensors 208 in FIG. 2 may represent a combination of one or more sensor units (containing different groups of sensors) and/or one or more individual sensors not integrated with other sensors into a sensor unit. The type of communication between a particular sensor 208 and the dissolution tester 212 depends on the type of sensor 208 and how the sensor 208 is coupled to the dissolution tester 212. For example, a sensor 208 configured to measure an attribute of a shaft 114 of the dissolution tester 212 may be physically coupled to the shaft 114, or may be mounted elsewhere at the vessel site 112 and have a component placed in contact with the shaft 114, or in non-contacting communication with the shaft 114 (e.g., an optics-based sensor 208). As another example, another type of sensor 208 may be inserted into a vessel 110 or mounted to another location of the dissolution tester 212. A particular sensor 208 may be integrated with the dissolution tester 212, or may be an external, portable sensor 208 that is mounted by the user to the dissolution tester 212 and is thereafter removable and transportable to another dissolution tester of the system 200. The sensors 208 may communicate with the user computer device 204 over one or more suitable communication links 220, which may be wired or wireless as noted above. A typical sensor 208 generally is a data acquisition device that produces data in the form of electrical signals in response to taking a measurement (such as by operation of a detector), and transmits the data to the user computing device 204 via the communication link 220. Typically, the data outputted by the sensor 208 is raw (unprocessed) data, but depending on the configuration of the sensor 208, the sensor 208 may process the data to some degree prior to transmission to the user computing device 204.

Examples of sensors 208 may include, but are not limited to, the following. In one example of a sensor 208, a centerline offset gauge may be provided to test for the degree to which a shaft 114 (e.g., a paddle shaft or basket shaft as in FIG. 1) of the dissolution tester 212 is aligned with the center axis of the vessel 110 in which the shaft 114 is inserted. In one example, the centerline offset gauge is mounted by the user to the shaft 114 and includes a linear optical encoder and a lateral plunger with a code strip readable by the linear optical encoder. The shaft 114 is lowered manually, or automatically by the drive unit 104 of the dissolution tester 212, into its normal operating position in the vessel 110, at which time the lateral plunger is spring-biased into contact with the inside surface of the vessel 110. The shaft 114 is rotated one full revolution and the linear optical encoder reads the code strip. Deviation of the shaft 114 from the centerline will be indicated by displacement of the lateral plunger as detected by the encoder's reading of the code strip. In another example of a sensor 208, a paddle/basket height gauge may be provided to measure the height of a paddle 124 or basket (FIG. 1) from the inner apex of the bottom of the vessel 110 in which the shaft 114 is inserted. In one example, the paddle/basket height gauge is mounted by the user to the shaft 114 and includes a linear optical encoder and a vertical plunger with a code strip readable by the linear optical encoder. To locate the true central bottom of the vessel 110, a stainless steel ball in placed in the vessel 110 and allowed to come to rest at the bottom. The shaft 114 is lowered manually or automatically into its normal operating position in the vessel 110, at which time the vertical plunger is spring-biased into contact with the top of the stainless steel ball. The position of the vertical plunger is read by the linear optical encoder and correlated to the height of the paddle 124 or basket from the bottom of the vessel 110. Examples of the centerline offset gauge and the paddle/basket height gauge include those provided with the above-noted VK 5010™ product and those disclosed in U.S. Pat. Nos. 6,434,847; 6,474,182; and 6,546,821, all assigned to the assignee of the present disclosure.

It will be understood, however, that the foregoing configurations for sensors 208 are non-limiting examples, and other configurations may be utilized. For example, a sensor 208 for measuring shaft centerline or shaft location in a vessel 110 may have a non-contacting (e.g., optical) configuration and may not require the shaft 114 to be rotated.

In an additional example of a sensor 208, a wobble gauge may be provided to measure the amount by which a shaft 114 (FIG. 1) of the dissolution tester 212 wobbles when rotated, i.e., deviates from a straight vertical line while rotating. In one example, the wobble gauge may have a design similar to a micrometer. The wobble gauge is mounted to a bracket surrounding the vessel 110 in which the shaft 114 is inserted and includes a plunger that extends into contact with the shaft 114. During rotation of the shaft 114, non-zero translation (movement perpendicular to the axis of the shaft 114) of the plunger indicates wobbling. In another example of a sensor 208, a tachometer sensor may likewise be mounted to a bracket surrounding the vessel 110 in which the shaft 114 is inserted, and configured to detect rotations per minute (RPM) of the shaft 114. In one example, the tachometer includes a magnetic sensor that reads a magnet affixed to a clip mounted to (and thus rotatable with) the shaft 114. In another example of a sensor 208, an electronic level sensor may be provided to measure the levelness (in degrees) of the vessel plate 106 and/or the drive unit 104 (FIG. 1) of the dissolution tester 212 and, simultaneously, the perpendicularity of each shaft 114 of the dissolution tester 212. For this purpose, the level sensor may be placed on the upper surface of the vessel plate 106 or on the upper surface of the drive unit 104. In another example of a sensor 208, a vibration sensor may be provided to measure, in three dimensions (i.e., X, Y and Z axes), vibrations generated by the dissolution tester 212 during operation. For this purpose, the vibration sensor may be placed on the upper surface of the vessel plate 106 or on the upper surface of the drive unit 104. The level sensor and the vibration sensor may be operated to take measurements while the shafts 114 of the dissolution tester 212 are in their normal operating positions in their respective vessels 110 and rotating at a predefined speed, and while each vessel 110 contains a predefined volume of a predetermined liquid. In another example of a sensor 208, a temperature probe may be provided to measure the temperature of the dissolution medium in a particular vessel 110 of the dissolution tester 212 and/or the temperature of the water bath 108 (if provided) in which the vessels 110 are immersed. For this purpose, the temperature probe may be inserted into a given vessel 110 or into the water bath 108 (FIG. 1). The wobble gauge, tachometer sensor, level sensor, vibration sensor and temperature probe may have any suitable designs, examples of which are provided with the above-noted QAII™ product. Again, however, it will be understood that the foregoing configurations for sensors 208 are non-limiting examples, and other configurations may be utilized. Additional examples of sensors 208 are described in U.S. patent application Ser. No. 12/905,806, titled METHODS AND APPARATUS FOR ACQUIRING PHYSICAL MEASUREMENTS RELATING TO A VESSEL AND A SHAFT WITHIN A VESSEL, filed Oct. 15, 2010, the content of which is incorporated by reference herein in its entirety.

As illustrated in FIG. 2, the user computing device 204 may be placed in signal communication with a network 224 over a wired or wireless communication link 228. To communicate with the network 224, the user computing device 204 may include various communication components understood by persons skilled in the art, such as a modem, router, hub, a network interface (e.g., an Ethernet or TCP/IP card), a communications port (e.g., serial, parallel, USB, RS232, RS422, IEEE 488, etc.), a PCMCIA card, PC card, communications interface software, and software for implementing network protocols (e.g., Ethernet or TCP/IP, Local Area Network or LAN, virtual LAN or vLAN, Wide Area Network or WAN, a metaframe technology arrangement with thin clients such as Citrix, etc.). Moreover, the system 200 may include or be part of a laboratory information management system (LIMS). The user computing device 204 may communicate with a remote computing device such as a database server 232 via the network 224 and appropriate communication links 228 and 236.

Additionally, one or more remote computing devices 240, 244 may communicate with the database server 232 and the local user computing device 204 over the network 224 via appropriate communication links 248, 252. Such computing devices 240, 244 are “remote” in the sense that they are typically situated such that they require the network 224 for communication with the local user computing device 204, and are not being utilized in conjunction with the sensors 208 to acquire measurement data from the particular dissolution tester 212 illustrated in FIG. 2, as that data acquisition task is being performed by the local user computing device 204. The local user computing device 204, the remote computing devices 240, 244 and the database server 232 may be utilized by coworkers of the same enterprise and located in the same facility or in different facilities. The enterprise may operate multiple dissolution testers (in addition to the illustrated dissolution tester 212) in the same facility or in different facilities. A local user computing device 204 may be dedicated for operation with each respective dissolution tester. Alternatively, one user computing device 204 may be utilized for acquiring measurement data from a group of dissolution testers located in the same facility or in the same laboratory of a facility. For instance, a user may couple a selected user computing device 204 and sensors 208 to a corresponding dissolution tester 212, acquire measurement data, and subsequently transport the same user computing device 204 (and optionally the same sensors 208) to another dissolution tester to acquire measurement data from that other dissolution tester.

The database server 232 may include database software 256 stored in memory. The database server 232 is configured for executing instructions of the database software 256 to create and maintain a database 260 that stores data records in an organized manner and provides access to the data records by the local user computing device 204 and the remote computing devices 240, 244 in a user-friendly manner and in a secured manner as desired. In one advantageous example, the database 260 is a relational database as understood by persons skilled in the art. The data records include measurement data acquired by the sensors 208 during measurement of the physical parameters of the dissolution tester 212. In a given data acquisition procedure, the user couples the sensors 208 to the dissolution tester 212 and to the user computing device 204. Depending on the type of sensors 208 being utilized, one or more sensors may first be coupled to the dissolution tester 212 and operated to acquire data relating to certain physical parameters, and then decoupled so that one or more other sensors may be utilized to acquire data relating to other physical parameters, according to a desired sequence of data acquisition. In operation, each sensor 208 makes measurements, converts the measurements to electronic signals, and transmits the signals to the user computing device 204 over the communication link 220. The user computing device 204 arranges the data received from each sensor 208 employed into a data record and transmits the data record to the database server 232 via the network 224 and communication links 228, 236. The database server 232 then stores the data record in its database 260. As appreciated by persons skilled in the art, the user computing device 204 is configured for processing the data captured from the sensors 208 in any manner necessary for transmitting the data record to the database server 232, such as temporarily storing the data, formatting the data for transmission over the network 224, etc. Alternatively, the user computing device 204 may send the measurement data to the database server 232, and the database server 232 may be configured to arrange the data in an appropriate data record or files.

In addition to measurement data received from the sensors 208, the data record may contain various types of information associated with the evaluation of the dissolution tester 212 being performed at a given time. For example, the data record may include the time and date of the evaluation event, an identification of the dissolution tester 212 being evaluated by the sensors 208 (e.g., dissolution tester serial number), and for some types of measurements an identification of a particular component of the dissolution tester 212 being measured (e.g., shaft serial number, vessel serial number, basket serial number, etc.).

The data record may also include, for each type of measurement being taken by the sensors 208 and for each component of the dissolution tester 212 being measured (e.g., each shaft 114, each vessel 110, etc.), an indication as to whether the particular physical parameter is in compliance or is not in compliance with a particular standard. The standard may be one that has been promulgated by a regulatory agency such as the FDA or USP, or may be a more stringent standard adhered to by the industry of which the enterprise is a part, an internal standard of the enterprise, or an internal standard required by a customer of the enterprise. For this purpose, the user computing device 204 may include evaluation software (i.e., data processing software) 264 configured to compare the actual measurement data received from the sensors 208 with a set of predefined values derived from standards, determine whether or not the particular physical parameter is in compliance, and add the results of this determination to the data record being created for this instance of evaluation of the dissolution tester 212. The database 260 may be utilized to maintain a list of predefined standards, or “methods,” which may be created by the user for the purpose of evaluating whether the physical parameters of the dissolution tester 212 satisfy the standards. The evaluation software 264 may further be configured to ensure technical compliance with 21 CFR 11. By automatically determining compliance or qualification of the physical parameters of the dissolution tester 212, the user computing device 204 reduces user error in interpretation of the value recorded by the sensors 208.

Alternatively, the evaluation software 264 may reside in and be executed by the database server 232. In this case, the user computing device 204 may be configured more thinly (in terms of software and/or hardware) to primarily transmit the acquired data (preprocessing and/or formatting the data as necessary) to the database server 232 for full processing and analysis.

As noted above, it is required or preferred that certain tasks be performed by or at the dissolution tester 212 in preparation for making certain types of measurements or while certain types of sensors 208 are making measurements, such as filling the test vessels 110 with liquid, lowering the shafts 114 into the test vessels 110, and rotating the shafts 114. The evaluation software 264 may include instructions executed by the user computing device 204 to automate these tasks in addition to controlling the sensors 208, thereby reducing the number of manual steps required and reducing the risk of human error. The evaluation software 264 may manage these tasks by causing the user computing device 204 to send appropriate control signals to the dissolution tester 212 (e.g., to the drive unit 104) via the communication link 216 to command the dissolution tester 212 to perform such tasks. As examples, the user computing device 204 may prompt the user (such as by a displayed graphical user interface) to verify that the user has installed the paddle/basket height measurement device on a given shaft 114 under evaluation, and upon receiving verification cause the drive unit 104 to lower the shaft 114 into its corresponding vessel 110 and subsequently activate the paddle/basket height measurement device to acquire the height measurement. The user computing device 204 may guide the user through the full procedure for height measurement for each shaft 114 of the dissolution tester 212. Similarly, for each shaft 114, the user computer device 204 may prompt the user to verify that the user has installed the shaft centerline offset measurement sensor on the shaft 114, and upon receiving verification cause the drive unit 104 to lower the shaft 114 into its corresponding vessel 110 and subsequently rotate the shaft 114 while activating the shaft centerline offset measurement sensor to make measurements at predetermined intervals during the rotation. Also for each shaft 114, the user computing device 204 may cause the drive unit 104 to lower the shafts 114 into the vessels 110, then prompt the user to verify that the user has installed the tachometer at a given vessel position in contact with the corresponding shaft 114 as well as the magnetic clip on the shaft 114, and upon receiving verification cause the drive unit 104 to rotate the shaft 114 at one or more selected speeds and activate the tachometer to make the speed measurement(s). Also for each shaft 114 or basket, the user computing device 204 may cause the drive unit 104 to lower the shafts 114 into the vessels 110 (if not already lowered), then prompt the user to verify that the user has installed the wobble gauge at a given vessel position 112 and in contact with the corresponding shaft 114 (or in contact with the rim of the basket, depending on which type of measurement is being performed), and upon receiving verification cause the drive unit 104 to rotate the shaft 114 at a predetermined speed and activate the wobble gauge to make the wobble measurement(s). The user computing device 204 may control the dissolution tester 212 and communicate with the user in conjunction with the procedures required for any other types of measurements contemplated, such as shaft verticality, vessel plate levelness, vessel plate vibration, drive unit vibration, or others.

Through execution of the evaluation software 264 and interfacing by way of a displayed user interface, the user computing device 204 may enable the user to select which types of physical parameters of the dissolution tester 212 are to be measured during a given evaluation session and the sequence by which different types of measurements are to be made, and may guide the user through the full procedure required for each type of measurement to be made and sensor 208 to be utilized to ensure all measurements have been taken correctly. After measurement data has been acquired for a particular type of shaft evaluation, the user computing device 204 may command the drive unit 104 to raise the shafts 114 out of the vessels 110 in preparation for measurement of another shaft 114 or in preparation for another type of measurement to be made by another type of sensor 208. The user computing device 204 may also prompt the user to enter a serial number for the dissolution tester 212 being evaluated and a serial number for each shaft 114 or vessel position 112 being evaluated. The association of serialized information with the captured measurement data for a particular dissolution tester 212 reduces user error in data entry and transcription, particularly when the dissolution tester 212 is re-tested at regular intervals. The evaluation software 264 may also be configured to output information that assists the user in making the changes or adjustments necessary for bringing certain physical parameters back into compliance.

After a particular evaluation session has been completed for the dissolution tester 212, the user computing device 204 may be configured to print out the same items of information that are stored in the data records of the database 260 to a printer (not shown) communicating with the user computing device 204 or another computing device 240, 244 associated with the network 224, either automatically or in response to a command inputted by the user. Moreover, the evaluation software 264 of the user computing device 204 may be configured to interrogate the database server 232 and produce qualification records based on the values recorded from the sensors 208 to satisfy regulatory requirements for physical qualification of the dissolution tester 212 and any other dissolution tester evaluated by the user computing device 204. Interrogation and production of qualification records may be done automatically according to a schedule predetermined by the user or at any time in response to a command by the user. The evaluation software 264 may be configured to print out qualification certificates according to any desired format. Additionally, the evaluation software 264 may be configured to print out calibration labels that may then be affixed to the dissolution tester 212 after a successful mechanical qualification.

The dissolution tester 212 may be evaluated for compliance periodically according to a predetermined schedule, utilizing the same user computing device 204 and sensors 208. Each time the dissolution tester 212 is evaluated, a data record specific to that instance of evaluation may be created and stored by the database server 232 in the database 260. The evaluation software 264 may be configured to access the several data records stored in the database 260 corresponding to several evaluation sessions, and perform a trend analysis of the data so as to determine whether any physical parameter of the dissolution tester 212 is moving toward an out-of-specification value. In this manner, the evaluation software 264 may predict the maintenance requirements of the dissolution tester 212.

One or more user computing devices 204 of the system 200 may be utilized to evaluate other dissolution testers, thereby generating multiple data records for all dissolution testers 212 according to a predetermined schedule. Hence, it will be appreciated by persons skilled in the art that the architecture of the system 200 may be extended to centralize dissolution tester information across an entire enterprise, thereby providing improved management of all data, scheduling of maintenance and calibration for all dissolution testers 212, and the ability to perform trend analysis for all dissolution testers 212 of that enterprise. Moreover, remote access to the centralized database 260 by remote computing devices 240, 244 as well as the local user computing device 204 provides visibility of the recorded data to multiple remote users simultaneously and on demand at any time. The evaluation software 264 residing in any given user's computing device is able to produce reports detailing the current qualification status of any dissolution tester 212 in the database to simplify management of the qualification status of dissolution testers 212 throughout the enterprise. The evaluation software 264 may also be configured to transmit (such as by electronic mail) calibration reports, as well as reminders and schedules regarding maintenance and evaluation of dissolution testers 212, to any computing device networked with the system 200. Additionally, extensions to the software 264 may be made to simplify data consumption by an external LIMS, as well as to provide any level of network security and user authentication requirements desired.

Alternatively or additionally to the database 260 maintained by the database server 232, the user computing device 204 may itself include database software 268 stored in its local memory. In this case, the user computing device 204 may be configured for executing instructions of the database software 268 to create and maintain a local database 272 containing and organizing the above-described data records of captured sensor data and user-inputted data. The local database 272 maintained by the user computing device 204 (and other computing devices local to other dissolution testers) may be synchronized with the master database 260 maintained by the database server 232 on a periodic basis. Alternatively, the local database 272 of the local computing device 204 may be utilized as the final repository for the data.

It thus can be seen that the system described herein is able to capture physical parameters obtained from a single dissolution tester or multiple dissolution testers via electronic sensors and provide a centralized view of these parameters. The system enables automated data collection, which greatly reduces transcription errors, and enables timely verification of transcribed results. The use of a database and other computer-related components provide a secure method of managing laboratory data, and the centralization of the data acquisition and management process improves overall quality control of the dissolution testing activities of an enterprise. As demonstrated above, captured data can be easily trended to predict maintenance requirements for a particular dissolution tester if the trend is moving toward a non-compliant value. Moreover, certain actions required of a user in a conventionally manual environment may now be automated. The system may be easily integrated with existing systems of an enterprise, such as a LIMS or other information management environment.

It will be understood that various aspects or details of the invention may be changed without departing from the scope of the invention. Furthermore, the foregoing description is for the purpose of illustration only, and not for the purpose of limitation—the invention being defined by the claims.

What is claimed is:

1. A method for acquiring and managing measurement data relating to operating parameters of a dissolution tester, the dissolution tester including a vessel support plate, a plurality of vessels mounted to the vessel support plate, a drive unit, and a plurality of shafts movable by the drive unit into the respective vessels and rotatable by the drive unit, the method comprising:
(a) measuring a plurality of operating parameters of the dissolution tester by operating a plurality of sensors communicating with the dissolution tester;
(b) transmitting the measured operating parameters from the sensors to a user computing device communicating with the sensors;
(c) determining whether the measured operating parameters transmitted to the user computer device are in compliance or non-compliance with one or more standards, by comparing the measured operating parameters with a plurality of corresponding predefined values; and
(d) storing the measured operating parameters and indications of compliance or non-compliance of each measured operating parameter as a data record in a memory communicating with the user computing device.

2. The method of claim 1, wherein the determining is done by operating the user computing device and the memory is local to the user computing device.

3. The method of claim 1, wherein the determining is done by operating the user computing device and the memory is located at a remote computing device communicating with the user computing device, and storing comprises transmitting the measured operating parameters and indications of compliance or non-compliance of each measured operating parameter to the remote computing device.

4. The method of claim 1, comprising transmitting the data record to another computing device situated remotely from the user computing device and remotely from the memory.

5. The method of claim 1, further comprising transmitting the measured operating parameters from the user computing device to a remote computing device, wherein the determining is done by operating the remote computing device and the memory in which the data record is stored.

6. The method of claim 1, comprising repeating steps (a)-(d) one or more times, wherein a plurality of data records are stored in the memory, each data record corresponding to a different time at which the plurality of operating parameters were measured, and further comprising interrogating the data records and determining whether one or more of the operating parameters are trending toward non-compliance and, based on the trending determination, determining a maintenance schedule for the dissolution tester.

7. The method of claim 1, comprising repeating steps (a)-(d) for one or more additional dissolution testers, wherein a plurality of data records are stored in the memory, each data record corresponding to a different dissolution tester for which the plurality of operating parameters were measured.

8. The method of claim 7, comprising, for each dissolution tester, repeating steps (a)-(d) one or more times, wherein a plurality of data records are stored in the memory, each data record corresponding to a different time at which the plurality of operating parameters were measured for each dissolution tester, and further comprising interrogating the data records and determining, for each dissolution tester, whether one or more of the operating parameters are trending toward non-compliance and, based on the trending determination, determining a maintenance schedule for the dissolution testers.

9. The method of claim 1, wherein the plurality of operating parameters comprise, for each shaft, a first shaft parameter and a second shaft parameter, and further comprising: while measuring the first shaft parameter, rotating the shaft at a first predetermined speed value by transmitting a first command from a user computing device to the drive unit; and while measuring the second shaft parameter, rotating the shaft at a second predetermined speed value by transmitting a second command from the user computing device to the drive unit.

10. The method of claim 9, wherein the first shaft parameter and the second shaft parameter are selected from the group consisting of shaft rotational speed and shaft wobble, shaft rotational speed and shaft centerline, and shaft wobble and shaft centerline.

11. The method of claim 1, wherein the plurality of operating parameters measured comprises operating parameters selected from the group consisting of shaft wobble, shaft rotational speed, shaft centerline, shaft height in a vessel, temperature of a liquid contained in a vessel, vessel plate levelness, drive unit levelness, vessel plate vibration, drive unit vibration, and combination of two or more of the foregoing.

12. The method of claim 11, comprising, while measuring one or more of the additional operating parameters, rotating the shaft at one or more respective, predetermined speed values by transmitting one or more respective commands from the user computing device to the drive unit.

13. The method of claim 1, comprising, if it is determined that a measured operating parameter is in non-compliance, generating an output at the user computing device that includes information for the user relating to a procedure for adjusting the dissolution tester so as to bring the operating parameter into compliance.

14. The method of claim 1, comprising, prior to measuring at least one of the operating parameters, raising or lowering at least one of the shafts by transmitting a command from the user computing device to the drive unit.

15. The method of claim 1, comprising, prior to measuring at least one of the operating parameters, flowing a liquid into at least one of the vessels by transmitting a command from the user computing device to the drive unit.

16. A system for acquiring and managing measurement data relating to operating parameters of a dissolution tester, the system comprising:
a dissolution tester comprising a vessel support plate, a plurality of vessels mounted to the vessel support plate, a drive unit, and a plurality of shafts movable by the drive unit into the respective vessels and rotatable by the drive unit;
a plurality of sensors configured for operative coupling to the dissolution tester for measuring a plurality of respective operating parameters of the dissolution tester;
a memory; and
a user computing device in signal communication with the dissolution tester and with the plurality of sensors, wherein the user computing device is configured for performing an evaluation comprising:
receiving a plurality of measured operating parameters generated by the respective sensors;
determining whether the measured operating parameters are in compliance or non-compliance with one or more standards, by comparing the measured operating parameters with a plurality of corresponding predefined values; and
storing the measured operating parameters and indications of compliance or non-compliance of each measured operating parameter as a data record in the memory.

17. The system of claim 16, comprising a database server situated remotely from the user computing device and communicating with the user computing device over a communication link, wherein the memory is located with the database server.

18. The system of claim 16, wherein the user computing device is configured for repeating the evaluation one or more times, storing one or more data records corresponding to the respective evaluations in the database memory, accessing the data records in the memory, determining whether one or more of the measured operating parameters are trending toward non-compliance and, based on the trending determination, determining a maintenance schedule for the dissolution tester.

19. The system of claim 16, comprising one or more remote computing devices situated remotely from the user computing device and the memory and configured for accessing the data records remotely over one or more respective communication links.

20. The system of claim 16, comprising one or more additional dissolution testers, wherein the user computing device is configured for performing the evaluation on each dissolution tester and storing one or more data records corresponding to the respective evaluations in the memory.

21. The system of claim 16, wherein the plurality of sensors comprises a first sensor configured for measuring a first shaft parameter and a second sensor configured for measuring a second shaft parameter, and the user computing device is further configured for: while the second sensor measures the second shaft parameter, transmitting a second command to the drive unit to rotate the shaft at a second predetermined speed value; and while the second sensor measures the second shaft parameter, transmitting a second command to the drive unit to rotate the shaft at a second predetermined speed value.

22. The system of claim 16, wherein the plurality of sensors is selected from the group consisting of a sensor configured for measuring shaft rotational speed, a sensor configured for measuring shaft wobble, a sensor configured for measuring shaft centerline, a sensor configured for measuring shaft height in a respective vessel, a sensor configured for measuring temperature of a liquid contained in a respective vessel, a sensor configured for measuring vessel plate levelness, a sensor configured for measuring drive unit levelness, a sensor configured for measuring vessel plate vibration, a sensor configured for measuring drive unit vibration, and a combination of two or more of the foregoing.

23. The system of claim 16, wherein the user computing device is configured for transmitting a command to the drive unit while performing the evaluation, wherein the command is selected from the group consisting of a command to raise at least one of the shafts out from a respective vessel, a command to lower at least one of the shafts into a respective vessel, a command to flow a liquid into at least one of the vessels, a command to prompt a user to input an identification of the dissolution tester or a component thereof being measured, and a combination of two or more of the foregoing.
